(19) United States
(12) Patent Application Publication
(10) Pub. No.: US 2012/0143249 A1
(43) Pub. Date: Jun. 7, 2012

(54) CLOSURE DEVICE

(75) Inventors: Mark L. Jenson, Greenfield, MN (US); Jason P. Hill, Brooklyn Park, MN (US)

(73) Assignee: Boston Scientific Scimed, Inc., Maple Grove, MN (US)

(21) Appl. No.: 13/268,142

(22) Filed: Oct. 7, 2011

Related U.S. Application Data
(60) Provisional application No. 61/419,553, filed on Dec. 3, 2010.

(51) Int. Cl. A61B 17/04; B32B 38/00; B32B 37/14

(52) U.S. Cl. 606/232; 606/144; 156/293

(57) ABSTRACT

The present disclosure provides closure devices for closing an opening in a body lumen, systems for closing an opening in a body lumen, and methods of making a closure device for closing an opening in a body lumen. The closure device may include a plug, an anchor, a cinch button, and a suture coupled to the anchor, the suture having a plurality of elongate filaments with a predefined cutting segment for cutting the suture, where the plurality of elongate filaments of the predefined cutting segment are bonded together to prevent their relative motion.

### PRIORITY INFORMATION

This application claims priority to U.S. Provisional Application No. 61/419,553 filed on Dec. 3, 2010, the specification of which is incorporated herein by reference.

### FIELD OF DISCLOSURE

Embodiments of the present disclosure are directed toward closure devices; more specifically, embodiments are directed toward closure devices for closing an opening in a body.

### BACKGROUND

Arteriotomy closure after diagnostic and/or interventional catheterization procedures has been addressed by a number of devices in addition to manual compression.

For a diagnostic and/or interventional catheterization procedure, such as a coronary procedure, a small gauge needle may be introduced through a patient's skin to a target blood vessel, such as the femoral artery in the region of the patient's groin. The needle forms a puncture, i.e., an arteriotomy, through the blood vessel wall. A guide wire may then be introduced through the needle, and the needle withdrawn over the guide wire. An introducer-sheath may be next introduced over the guide wire, and the sheath and guide wire may be left in place to provide access during the procedure. Examples of procedures include diagnostic procedures such as angiography, ultrasonic imaging, and the like, and interventional procedures, such as angioplasty, atherectomy, stent placement, cardiac valve procedures, laser ablation, graft placement, and the like.

After the procedure is completed, the catheters, guide wire, and introducer-sheath are removed, and it is necessary to close the arteriotomy to provide hemostasis, i.e., stop blood loss, and allow healing.

### SUMMARY

One or more embodiments of the present disclosure include a closure device for closing an opening in a body lumen. The closure device may include a plug, an anchor, a cinch button, and a suture coupled to the anchor. In one embodiment, the suture includes a plurality of elongate filaments with a predefined cutting segment for cutting the suture, wherein the plurality of elongate filaments of the predefined cutting segment are bonded together to prevent their relative motion.

One or more embodiments of the present disclosure include a system for closing an opening in a body lumen. The system may include an insertion sheath, a device sheath releasably housed in the insertion sheath, a closure device, and a push member.

One or more embodiments of the present disclosure include a method of making the closure device for closing an opening in a body lumen. The method may include providing a suture having a plurality of elongate filaments, forming a predefined cutting segment along the suture by bonding the plurality of elongate filaments to prevent their relative motion, embedding the suture into the anchor to couple the suture to the anchor, positioning a plug over a length of the suture, and positioning a cinch button along the suture to couple the plug to the anchor.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 illustrates a closure device according to an embodiment of the present disclosure.

FIG. 2 illustrates a closure device according to an embodiment of the present disclosure.

FIG. 3A illustrates a suture according to an embodiment of the present disclosure.

FIG. 3B illustrates a cross-sectional view of the suture of FIG. 3A along line 3B-3B.

FIG. 3C illustrates a cross-sectional view of the predefined cutting segment of FIG. 3A along line 3C-3C.

FIG. 4 illustrates a suture according to an embodiment of the present disclosure.

FIG. 5 illustrates a cross-sectional view of the suture of FIG. 4 along line 5-5.

FIGS. 6A-6B illustrate cross-sectional views of the predefined cutting segment of FIG. 4 along line 6A-B-6A-B.

FIG. 7A illustrates a suture according to an embodiment of the present disclosure.

FIG. 7B illustrates a cross-sectional view of the suture of FIG. 7A along line 7B-7B.

FIGS. 8A-8B illustrate cross-sectional views of the predefined cutting segment of FIG. 7A along line 8A-B-8A-B.

FIG. 9 illustrates a system for closing an opening in a body lumen according to an embodiment of the present disclosure.

FIG. 10 illustrates the system for closing an opening in a body lumen disposed within the insertion sheath according to an embodiment of the present disclosure.

The Figures are not to scale.

### DETAILED DESCRIPTION

Embodiments of the present disclosure are directed to a closure device for closing an opening in a body lumen, a system that includes the closure device, and a method of making the closure device. For the embodiments, the closure device may include a plug, an anchor, a cinch button, and a suture. The suture is coupled to the anchor and extends through the plug. The suture has a plurality of elongate filaments with a predefined cutting segment for cutting the suture. The plurality of elongate filaments of the predefined cutting segment are bonded together to prevent their relative motion to facilitate cutting the suture.

Anchor-plug-cinch devices have been employed for arteriotomy closure procedures, e.g., closing an opening in a body lumen. The anchor can be disposed within a body lumen of a vessel and the plug can be disposed adjacent an exterior surface of the vessel wall or partially within the body lumen. The anchor and plug are cinched together with body vessel wall tissue pinched between the plug and the anchor. Once cinched together, the suture can be cut to implant the closure device. As used herein “suture” refers to a multifilament, multi-fibril, or multi-threadlike structure that is braided, twisted, or intertwined. For anchor-plug-cinch devices, multifilament sutures can be desirable for a variety of reasons. For example, multifilament sutures can be more flexible than monofilaments and provide additional options for coupling the suture to the anchor.

Cutting the suture to implant the closure device can leave a distance between the suture and the patient's skin. Maximizing the distance between the suture and the skin of the patient can reduce infections by helping to remove an access path from outside the body to the tissues underneath the skin of the patient. Moreover, maximizing the distance between the suture and the skin of the patient can further help reduce irritation.

One previous approach for cutting the suture requires the physician to manually cut the suture to implant the closure device by manually pulling tension on the suture, manually depressing the skin, and manually cutting the suture with a manual cutting mechanism, e.g., a scalpel or scissors. The suture is cut close to the depressed skin so that when the skin is released, an end of the suture is underneath the surface of the skin. However, manually depressing the skin to cut the suture can limit the distance between the skin of the patient and the end of the suture.

One approach to maximize the distance between the skin and the end of the cut suture can include using a remote cutting mechanism, which can cut the suture farther underneath the skin as compared to the manual method. The remote cutting mechanism can also be automatic, which cuts the suture when the closure device has been deployed. However, using a remote cutting mechanism for cutting multifilament sutures can be unreliable. For example, portions of the multifilament suture can deform into clearances provided in the automatic cutting mechanism. As used herein “clearances” refer to spaces and/or potential spaces between moving parts of the automatic cutting mechanism. For illustration, when using common scissors to cut a filament, the filament can slightly displace the cutting components, i.e., shearing blades, and separate them.

Cutting a multifilament structure, e.g. a suture having a plurality of elongate filaments, can increase the difficulty of cutting as compared to a monofilament because the plurality of filaments can move and align such that there is a small separation between the cutting components, which can foil the cutting. Therefore, displacement of the cutting components of the remote cutting mechanism can prevent proper cutting of the multifilament suture. Even a slight displacement of the cutting components can increase the risk of not properly cutting the multifilament suture. Additionally, even if some of the filaments of the multifilament suture are successfully cut, one or more filaments can slide between the cutting components and not be properly cut.

Portions of the multifilament suture that deform into the clearances can result in failure to cut all filaments of the multifilament suture. If a sufficient number of filaments remain uncut, the suture cannot be separated and the excess length of suture will not be removed. Additionally, portions of deployment and implantation systems may remain attached, requiring further procedures to remove them from the patient.

Previous approaches using a remote cutting mechanism to cut multifilament sutures were limited to complex and expensive remote cutting mechanisms. The complex and expensive remote cutting mechanisms were used because they included smaller and/or fewer clearances for the multifilament suture to deform into and could more reliably cut the multifilament sutures as compared to the less complex and less expensive remote cutting mechanisms.

The present disclosure describes embodiments of a closure device that provides a suture having a plurality of elongate filaments with a predefined cutting segment for cutting the suture. The plurality of elongate filaments of the predefined cutting segment are bonded together to prevent their relative motion and is more rigid than the remainder of the suture. Bonding the elongate filaments of the predefined cutting segment together can minimize the number of plurality of elongate filaments that deform into clearances of a remote cutting mechanism. Since the predefined cutting segment is more rigid, it can be cut more easily and reliably using less complicated and less expensive remote cutting mechanisms. Therefore, the closure device of the present invention allows the suture to be cut with the remote cutting mechanism, which can cut the suture farther beneath the skin of the patient versus manually cutting the suture.

As used herein, “a,” “an,” “the,” “at least one,” and “one or more” are used interchangeably. The term “and/or” means one, one or more, or all of the listed items. The recitations of numerical ranges by endpoints include all numbers subsumed within that range (e.g., 1 to 5 includes 1, 1.5, 2, 2.75, 3, 3.80, 4, 5, etc.).

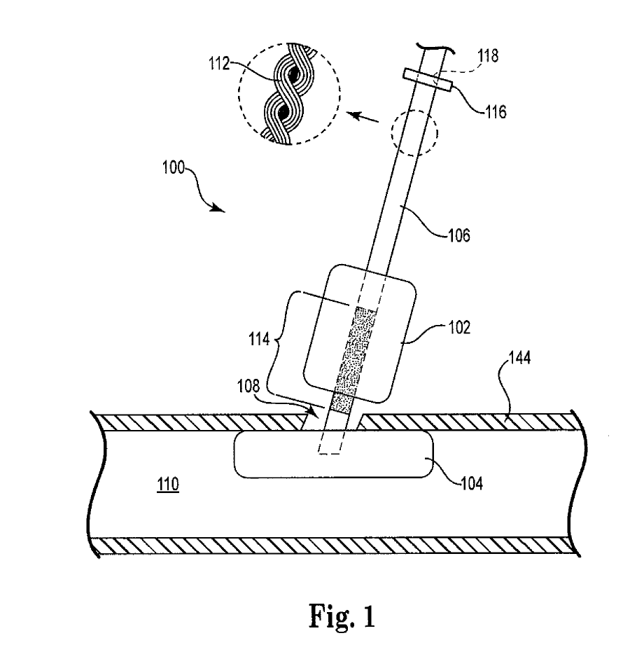

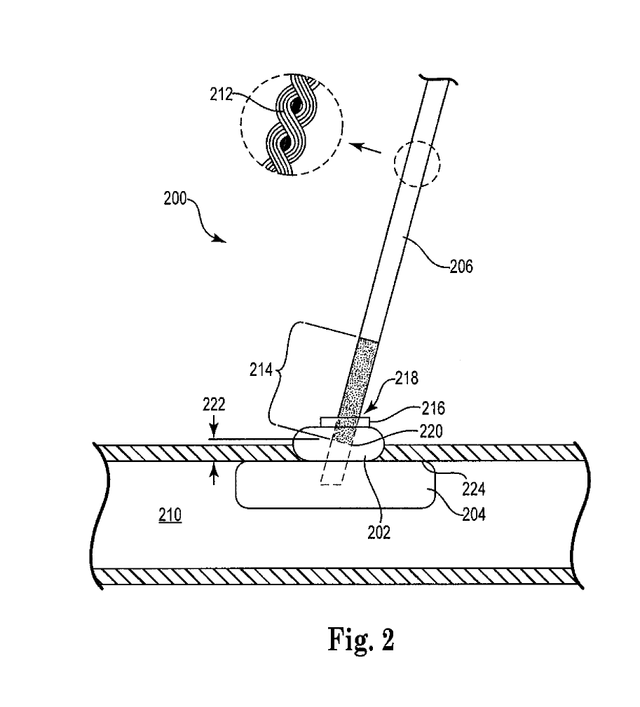

The figures herein follow a numbering convention in which the first digit or digits correspond to the drawing figure number and the remaining digits identify an element in the drawing. Similar elements between different figures may be identified by the use of similar digits. For example, 106 may reference element “06” in FIG. 1, and a similar element may be referenced as 206 in FIG. 2.

Various non-limiting embodiments of the present disclosure are illustrated in the figures. Generally, the closure device can be implanted to close an opening into a body lumen.

FIG. 1 illustrates a closure device 100 according to an embodiment of the present disclosure. The closure device 100 includes a plug 102, an anchor 104, and a suture 106. The closure device 100 in FIG. 1 illustrates the anchor 104 and plug 102 prior to being cinched together, i.e., the plug 102 is in an undeformed state. For one or more embodiments, the suture 106 is coupled to the anchor 104 such that the suture 106 is secured to the anchor 104 and extends through the plug 102. As seen in FIG. 1, the suture 106 is embedded into the anchor 104 and is positioned through the plug 102 such that a portion of the suture 106 passes through an opening 108 into a body lumen 110. The anchor 104 can be disposed within the body lumen 110 of a vessel and the plug 102 can be disposed adjacent an exterior surface of the vessel wall 144. Additionally, the plug 102 can be positioned across the opening 108.

For one or more embodiments, the suture 106 has a plurality of elongate filaments 112. As seen in FIG. 1, the plurality of elongate filaments 112 are illustrated as being braided. However, the plurality of elongate filaments 112 can be parallel, twisted, intertwined, or combinations thereof.

For one or more embodiments, the suture 106 includes a predefined cutting segment 114 for facilitating cutting of the suture 106. The plurality of elongate filaments 112 of the predefined cutting segment 114 are bonded together to prevent their relative motion. The predefined cutting segment 114 is more rigid than the remainder portion of the suture 106, which helps facilitate cutting the suture 106 during implantation of the closure device 100.

For one or more embodiments, the closure device 100 includes a cinch button 116 having an aperture 118. The suture 106 extends from the anchor 104 through the plug 102 and through the aperture 118 of the cinch button 116. For one or more embodiments, the cinch button 116 can be moved longitudinally along the suture 106 to allow for a compressive axial force to be applied to the plug 102. For example, FIG. 1 illustrates an embodiment in which the closure device 100 is positioned across the opening 108 into the body lumen 110, but is in an undeformed state. To achieve the deformed state of the closure device 100, the cinch button 116 can be advanced along the suture 106 towards the anchor 104, where it comes into contact with the plug 102.

FIG. 2 provides an illustration of one embodiment of the plug 202 in a deformed state. As seen in FIG. 2, the cinch button 216 can be advanced along the suture 206 to deform, e.g., compress, the plug 202. The cinch button 216 can hold the plug 202 in the deformed state using a variety of techniques. One technique includes holding the plug 202 in the deformed state by a friction fit between the cinch button 216 and the suture 206. For one or more embodiments, the cross-sectional area of the suture 206 and the cross-sectional area of the aperture 218 of the cinch button 216 can be sufficiently different to provide for a friction fit that will reduce the chances that the cinch button 216 will allow the plug 102 to change from the deformed state, i.e., undeform. In one or more embodiments, the suture 206 and the cinch button 216 can be configured to include a one-way mechanism. The one-way mechanism can prevent the cinch button 216 from reversibly moving along the suture 206 once the cinch button 216 has passed the one-way mechanism. One-way mechanisms can include, but are not limited to, a ratchet system, barbs, and combinations thereof. Other one-way mechanisms can be implemented such that the cinch button 216 does not reversibly move along the suture 206 once the cinch button 216 deformed the plug 202.

As discussed herein, the plurality of elongate filaments 212 of the predefined cutting segment 214 are bonded together to prevent their relative motion. Preventing the relative motion of the plurality of elongate filaments 212 of the predefined cutting segment 214 can increase the rigidity of the predefined cutting segment 214, which can facilitate cutting of the suture 206. For one or more embodiments, a rigidity of the predefined cutting segment 214 is greater than a rigidity of the suture 206 that is not the predefined cutting portion 214.

By increasing the rigidity of the predefined cutting segment 214, the number of plurality of elongate filaments 212 that may deform into clearances in the remote cutting mechanism can be minimized. As discussed herein, minimizing the number of plurality of elongate filaments 212 that deform into clearances of the remote cutting mechanism can allow the remote cutting mechanism to be configured with clearances while still reliably cutting the suture 206, i.e., successfully cutting a substantial portion of the plurality of elongate filaments 212. Remote cutting mechanisms configured with clearances can make the fabrication of the remote cutting mechanism easier and is less expensive. Utilizing the remote cutting mechanism can allow the distance between the suture and the skin of the patient to be maximized while reliably cutting the suture.

For one or more of the embodiments, a distal end 220 of the predefined cutting segment 214 can be positioned at a predetermined distance 222 from a surface 224 of the anchor 204. The distal end 220 of the predefined cutting segment 214 can be positioned within the plug 202 when the plug 202 is deformed (as illustrated in FIG. 2). However, other configurations are possible. For example, the distal end 220 of the predefined cutting segment 214 can be positioned within the aperture 218 of the cinch button 216. Additionally, the distal end 220 can be positioned adjacent to or proximal to the cinch button 216 such that the distal end 220 is not within the cinch button 216 or the plug 202. For one or more embodiments, the distal end 220 can be positioned within the anchor 204 such that the distal end 220 extends into the body lumen 210.

Various factors can affect where the distal end 220 of the predefined cutting segment 214 is positioned. For example, a thickness of the cinch button 216, a distance the plug 202 deforms, among other factors, can determine the position of the distal end 220 of the predefined cutting segment 214 along the suture 206.

As seen in FIG. 2, the length of the predefined cutting segment 214 can allow the predefined cutting segment 214 to extend across the cinch button 216 and into the lumen of the plug 202 when the plug 202 is deformed (as illustrated in FIG. 2). However, other configurations are possible. For example, the predefined cutting segment 214 can extend the entire length of the suture 206 or only a portion of the length of the suture 206. However, one consideration is that the predefined cutting segment 214 extends a sufficient length past a proximal surface of the cinch button 216 such that the remote cutting mechanism can cut the suture 206 at a point along the predefined cutting segment 214 when the plug 202 is in the deformed state.

The distance the plug 202 deforms can vary and can be based on the manufacture and materials being utilized for the plug 202. Additional considerations can include types of push rod actuation and actuation mechanism materials and tolerances, discussed herein. For example, the plug 102 in the undeformed state (as illustrated in FIG. 1) can have a length of about 2 centimeters (cm) and the plug 202 in the deformed state (as illustrated in FIG. 2) can have a length in a range of from 3 millimeters (mm) to 6 mm. Additionally, the type of remote cutting mechanism being used can have a known cutting position. Thus, one skilled in the art can determine the length of the predefined cutting segment 214 based on the variety of factors discussed herein.

For one or more embodiments, the length of the predefined cutting segment 214 can be within a range of from 1 mm to 10 mm. In some embodiments, the length can be less than 1 mm. Additionally, in some embodiments, the length can be greater than 10 mm.

As discussed herein, after cinching the plug 202 and anchor 204 together with the cinch button 216, i.e., the deformed state in FIG. 2, the suture 206 can be cut along the predefined cutting segment 214 to implant the closure device 200. Minimizing the length of the suture 206 that extends from the cinch button 216 after the suture 206 is cut can minimize tissue irritation underneath the skin of the patient and reduce infection. For example, the length of the remaining suture can be within a range of 0.5 mm to 5 mm. The remaining suture can include a portion of the predefined cutting segment or can be only the predefined cutting segment.

As discussed herein, the plurality of the elongate filaments of the predefined cutting segment are bonded together to prevent their relative motion. Preventing the relative motion of the plurality of filaments of the predefined cutting segment can increase the rigidity of the predefined cutting segment as compared to the rigidity of the suture that is not the predefined cutting portion to facilitate cutting of the suture. Bonding the plurality of filaments of the predefined cutting segment to prevent their relative motion can be accomplished by applying a bonding agent, compression, heat, a solvent, tension, and combinations thereof.

For one or more embodiments, the predefined cutting segment includes the bonding agent to bond the plurality of elongate filaments of the predefined cutting segment. As used herein “bonding agent” is a material that can fuse and/or join the plurality of elongate filaments of the predefined cutting agent as to prevent their relative motion. For one or more embodiments, the bonding agent can be selected from the group consisting of polymers, sugars, biological materials, adhesives, and combinations thereof.

Examples of polymers include, but are not limited to, polygycolic acid, polylactic acid, poly(lactic-co-glycolic acid), polyesters, polycaprolactone, polydioxanone, and combinations thereof. Additional examples of polymers include PEG, PEGylated materials such as PEGylated proteins, and related materials.

Sugar, as used herein, refers to carbohydrates including monosaccharides, disaccharides, oligosaccharides, and polysaccharides having, e.g. four (tetrose), five (pentose), six (hexose), seven (heptose), or more carbon atoms, and combinations thereof. Examples of monosaccharides include, but are not limited to, allose, altrose, glucose, mannose, gulose, idose, galactose, talose, ribose, arabinose, xylose, lyxose, erythrose, threose, glyceraldehyde, and combinations thereof. Examples of disaccharides include, but are not limited to, cellobiose, maltose, lactose, gentiobiose, sucrose, and combinations thereof. Examples of oligosaccharides and/or polysaccharides include, but are not limited to, cellulose, starch, amylase, amylopectin, glycogen, and combinations thereof.

Examples of adhesives include, but are not limited to, cyanoacrylates, biological adhesives, protein-based adhesives, starch-polyacrylic blend adhesives, thermoplastic polymer adhesives, 2-part polymer adhesives, bonding or strengthening agents, and combinations thereof. In addition to materials thought of as adhesives, materials used in the suture can be applied to bond, e.g., “glue”, the plurality of filaments together.

For one or more embodiments, the suture may include a biological material. Examples of biological materials include, but are not limited to, surgical gut, e.g. catgut, silk, and combinations thereof. For one or more embodiments, the biological material may be treated with a chromium salt solution to provide a chromatic suture material. For one or more embodiments, the suture may include protein material, derivatives, or synthetic analogs, such as collagen or modified collagen.

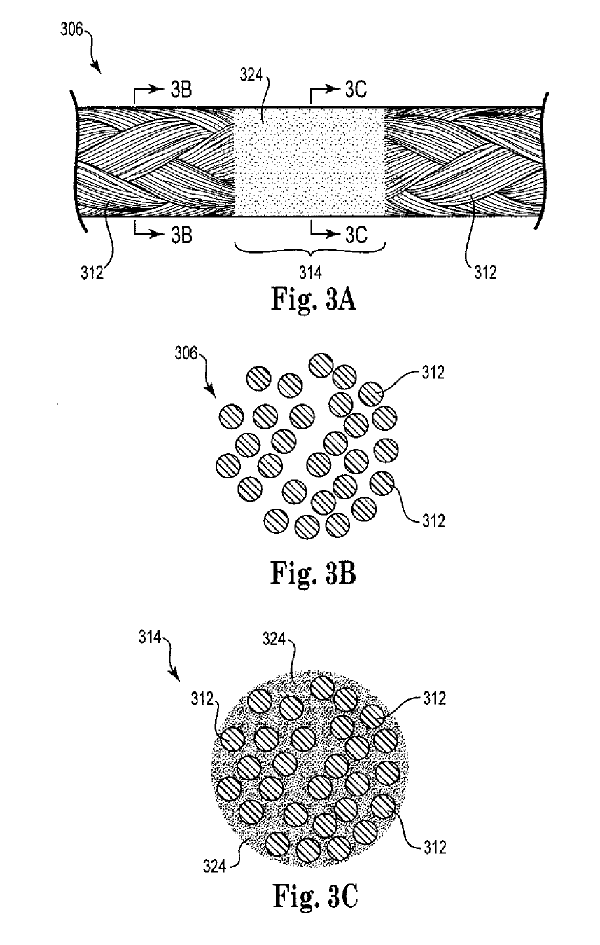

FIG. 3A illustrates a suture 306 according to an embodiment of the present disclosure. As discussed herein, the plurality of the elongate filaments 312 of the predefined cutting segment 314 are bonded together to prevent their relative motion. For one or more embodiments, the predefined cutting segment 314 includes a bonding agent 324. In one embodiment, the predefined cutting segment 314 has a substantially similar diameter as the portion of the suture 306 that is not the predefined cutting segment 314.

FIG. 3B illustrates a cross-sectional view of the suture 306 of FIG. 3A along line 3B-3B. As seen in FIG. 3B, the plurality of elongate filaments 312 of the suture 306 that are not the predefined cutting segment 314 define available space between the plurality of elongate filaments 312. The cross-section of the plurality of elongate filaments 312, as seen in FIG. 3B, are illustrated as circles, however, other cross-sectional shapes may be used. For example, the cross-section shape of the plurality of elongate filaments 312 can include, but not limited to, a polyhedron, ellipse, square, triangle, and combinations thereof.

FIG. 3C illustrates a cross-sectional view of the predefined cutting segment 314 of FIG. 3A along line 3C-3C. For one or more embodiments, the bonding agent 324 can be applied within the available space between the plurality of elongate filaments 312 of the predefined cutting segment 314. The bonding agent can be applied by application techniques including, but not limited to, spraying, dipping, coating, painting, co-extruding, misting, precipitating, and combinations thereof. Additional application techniques known in the art may also be used.

As seen in FIG. 3C, the bonding agent 324 is applied within the available space such that the diameter of the predefined cutting segment 314 is not substantially increased. That is, the diameter of the suture 306 in FIG. 3B is substantially the same as the diameter of the predefined cutting segment 314 in FIG. 3C.

For one or more embodiments, the bonding agent can be applied to an external surface of the predefined cutting segment to increase the diameter of the predefined cutting segment as compared to the suture that is not the predefined cutting segment. Additionally, the bonding agent can be applied both within the available space between the plurality of elongate filaments and along the external surface of the predefined cutting segment.

For one or more embodiments, the bonding agent can have a viscosity and relative surface energy that allows capillary action to occur between the bonding agent and the space between the plurality of elongate filaments of the predefined cutting segment. The capillary action can draw the bonding agent into the space between the plurality of elongate filaments of the predefined cutting segment to bond the plurality of elongate filaments of the predefined cutting segment together.

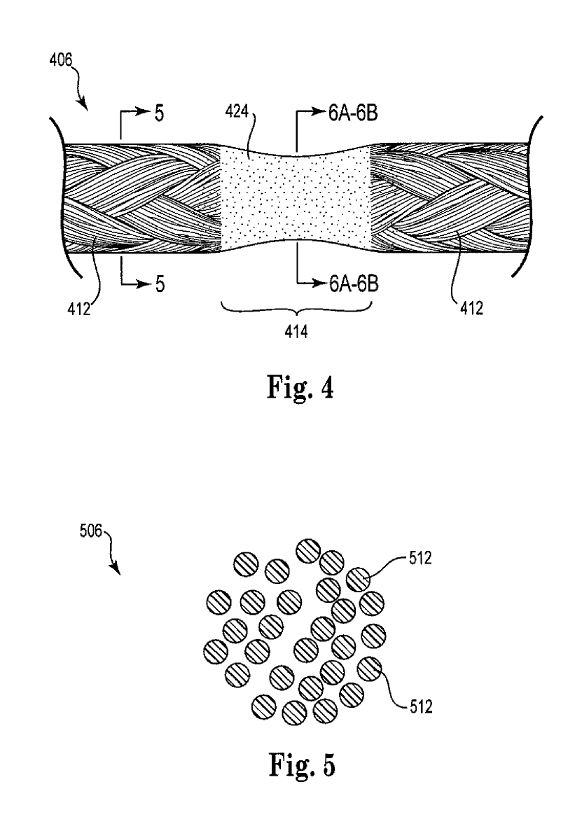

FIG. 4 illustrates a suture according to an embodiment of the present disclosure. As seen in FIG. 4, the diameter of the predefined cutting segment 414 is smaller than the diameter of the suture 406 that is not the predefined cutting segment 414.

FIG. 5 illustrates a cross-sectional view of the suture 406 of FIG. 4 along line 5-5. As seen in FIG. 5, the plurality of elongate filaments 512 define available space between each of the plurality of elongate filaments 512. The cross-sectional shape of the suture 506 is a circle; however, as discussed herein with reference to FIG. 3B, the cross-sectional shape of the suture can include other shapes.

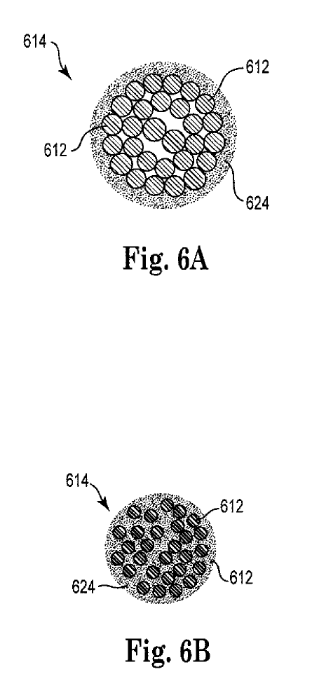

FIGS. 6A-6B illustrate cross-sectional views of embodiments of the predefined cutting segment 414 of FIG. 4 along line 6A-B-6A-B. In FIG. 6A, the space available between the plurality of elongate filaments 612 can be minimized by compression. Applying compression to the plurality of elongate filaments 612 can allow the plurality of elongate filaments 612 to contact each other, minimizing the available space between the plurality of elongate filaments 612. For one or more embodiments, once the plurality of elongate filaments 612 in contact via compression, the bonding agent, heat, and/or a solvent can be applied to bond the plurality of elongate filaments 612 together to prevent their relative movement. FIG. 6A illustrates an embodiment of the predefined cutting segment 614 where the plurality of elongate filaments 612 of the predefined cutting segment 614 are bonded together by compression and application of the bonding agent 624. As seen in FIG. 6A, the compression minimizes the space between the plurality of elongate filaments 612 while the bonding agent 624 is applied along an external surface of the compressed plurality of elongate filaments 612. The bonding agent 624 can be selected from materials as described herein. In one embodiments, the bonding agent 624 can be applied to the plurality of elongate filaments 612 followed by compression.

FIG. 6B illustrates an embodiment of the predefined cutting segment 614 where tension is applied to the plurality of elongate filaments 612 of the predefined cutting segment 614. Applying tension in combination with at least one of: the bonding agent, heat, and the solvent can bond the plurality of elongate filaments 612 of the predefined cutting segment 614 together. As seen in FIG. 6B, the plurality of elongate filaments 612 are bonded together by application of tension in combination with the bonding agent 624. The tension necks the plurality of elongate filaments 612. As used herein, “necks” is a mode of tensile deformation where relatively large amounts of strain localize disproportionately in a small region of the material and the local cross-sectional area decreases.

Thus, the cross-sectional area of the plurality of elongate filaments 612 of the predefined cutting segment 614 that are necked have a cross-sectional area that is less than the cross-sectional area of the plurality of elongate filaments that are not the predefined cutting segment. That is, the cross-sectional area of the plurality of elongate filaments 612 in FIG. 6B is less than the cross-section area of plurality of elongate filaments 512 in FIG. 5. As discussed herein, necking the plurality of elongate filaments 612 can be combined with at least one of: the bonding agent, heat, and the solvent to bond the plurality of elongate filaments 612 of the predefined cutting segment 614 together and prevent their relative motion.

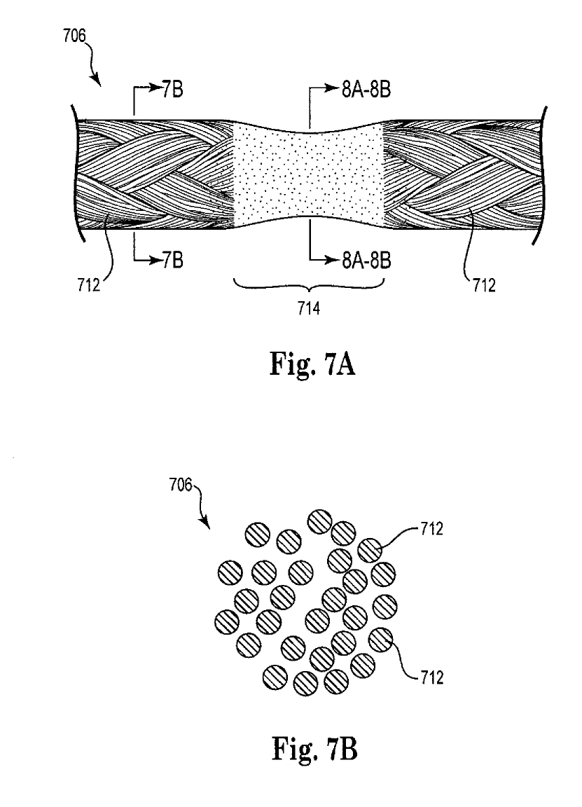

FIG. 7A illustrates an embodiment of the suture according to an embodiment of the present disclosure. As seen in FIG. 7A, the diameter of the predefined cutting segment 714 is smaller than the diameter of the suture 706 that is not the predefined cutting segment 714.

FIG. 7B illustrates a cross-sectional view of the suture along lines 7B-7B in FIG. 7A. As seen in FIG. 7B, the plurality of elongate filaments 712 define available space between each of the plurality of elongate filaments 712. The cross-sectional shape of the suture 706 is a circle; however, as discussed herein with reference to FIG. 3B, the cross-sectional shape of the suture can include other shapes.

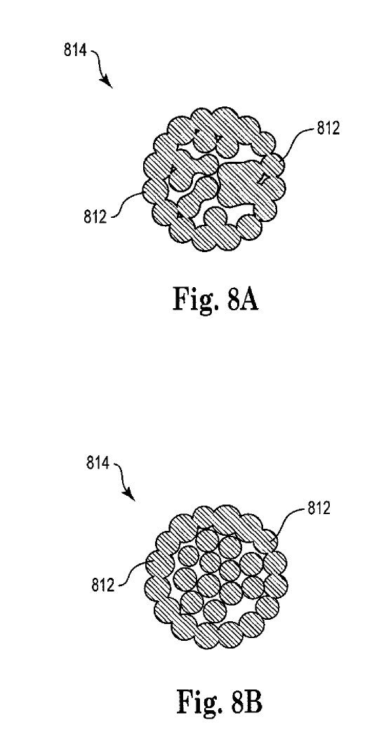

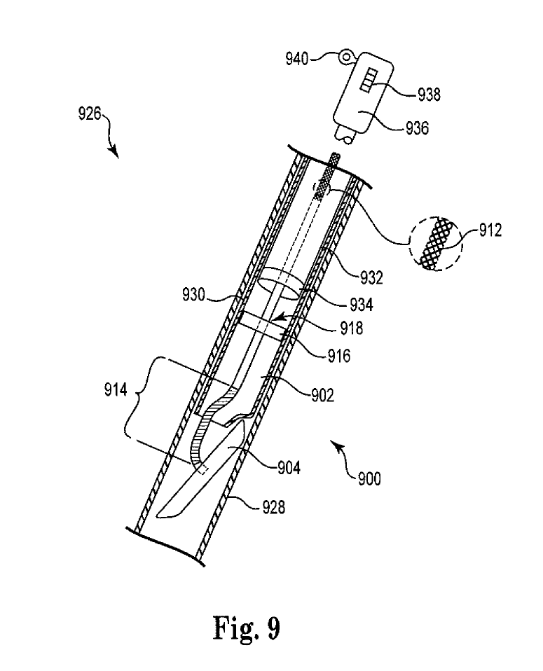

FIGS. 8A and 8B illustrate cross-sectional views of embodiments of the predefined cutting segment along line 8A-B-8A-B in FIG. 7A. FIG. 8A illustrates an embodiment of the predefined cutting segment 814 where the plurality of elongate filaments 812 of the predefined cutting segment 814 are bonded together by applying a solvent. The solvent can be applied by application techniques, as discussed herein. In one embodiment, the solvent can be a substance that interacts with the plurality of elongate filaments 812 of the predefined cutting segment 814. For example, the solvent can partially dissolve the plurality of elongate filaments 812 of the predefined cutting segment 814. Once the solvent is applied, the partially dissolved plurality of elongate filaments 812 can be allowed to dry, i.e., allow the solvent to evaporate, bonding the plurality of elongate filaments 812 together to prevent their relative motion. For one or more embodiments, bonding the plurality of elongate filaments 812 together by applying the solvent can be in combination with compression, heat, and/or the bonding agent.

FIG. 8B illustrates an embodiment of the predefined cutting segment 814 where the plurality of elongate filaments 812 of the predefined cutting segment 814 are bonded together by combination of compression and heat. As seen in FIG. 8B, the compression minimizes the space between the plurality of elongate filaments 812 while the application of heat can melt a portion of the plurality of elongate filaments 812. Melting a portion of the plurality of elongate filaments 812 together can bond the plurality of elongate filaments 812 of the predefined cutting segment 814 and prevent their relative motion.

Additionally, it is also possible to form the predefined cutting segment by melting substantially all of the plurality of elongate filaments together forming a single solid structure. For one or more embodiments, the heat can be applied after the compression of the plurality of elongate filaments. Alternatively, the heat can be applied before the compression or simultaneously. Application of heat can, e.g., be accomplished by ultrasonic welding, infrared heating, thermal conduction, or other thermal mechanisms.

For one or more embodiments, the solvent can be selected from the group consisting of dimethylformamide, dimethyl sulfoxide, hexane, tetrahydrofuran, toluene and combinations thereof. Additional solvents may also be used. Solvents can be used to dissolve at least a portion of the plurality of elongate filaments in the predefined cutting section, used to soften the surface of the filaments, to make the filaments tacky, or to chemically modify or cross-link the filaments, to aid in bonding the plurality of elongate filaments of the predefined cutting segment together.

As seen in FIGS. 5 and 7A, the predefined cutting segment is illustrated as having a decreased diameter as compared to the remainder of the suture that is not the predefined cutting segment. However, the diameter of the predefined cutting segment of FIGS. 5 and 7 can be configured to be substantially the same or greater than the remainder of the suture that is not the predefined cutting segment. For example, a sufficient amount of the bonding agent, as discussed herein, can be applied to the predefined cutting segment of FIGS. 5 and 7A to increase the diameter such that the diameter of the predefined cutting segment is substantially the same or greater than the remainder of the suture.

For one or more embodiments, the predefined cutting segment can include a monofilament portion. For example, the predefined cutting segment can be formed by fusing a monofilament suture between two ends of a suture having a plurality of elongate filaments, i.e., a multifilament suture.

For one or more embodiments, the suture can be formed from biodegradable materials. For example, the suture can be formed from biodegradable esters, sugars, biological materials, or protein-based materials such as those cited herein, and combinations thereof. The biodegradable suture may break down and be absorbed by the body. For example, the suture may biodegrade and be absorbed into the body in a period of time of 3 days to 180 days when deployed within the body lumen. For one or more embodiments, the suture may further include silk, collagen, medicinal materials, antibiotics, antimicrobials, inflammation modifiers, imaging enhancers, strengtheners, and combinations thereof.

For one or more embodiments, the anchor, as disclosed herein, may be biodegradable. For example, the anchor may degrade within a body in a period of time of 30 days to 120 days. For some applications, the anchor may degrade within the body in a period of time of 3 days to 180 days. For one or more embodiments, the anchor may be formed from polymers, sugars, biological materials, or protein-based materials, and combination thereof, as discussed herein.

For various applications, the anchor may have differing shapes. For one or more embodiments, the anchor may include one or more shapes including, but not limited to, polyhedron, sphere, cylinder, cone, and combinations thereof. For some applications, the anchor may have a first surface configured to appose the body lumen. For example, the first surface may be convex in relation to the anchor such that the convex first surface conforms to a concave surface of the body lumen. For some applications, the anchor may have a second surface configured to help minimize flow disturbances and/or flow separation within the body lumen. For example, the second surface may be canted, e.g., where a first end and a second end of the anchor have a thickness that is less than a thickness at the center of the anchor.

For various applications the anchor may have differing dimensions. For one or more embodiments, the anchor may have a length within a range of from of 1 mm to 25 mm. For example, the anchor may have a length of 5 mm to 18 mm, or 8 mm to 13 mm. For one or more embodiments, the anchor may have a width with in a range of from 1 mm to 8 mm. For example the anchor may have a width of 1 mm to 5 mm, or 1.5 mm to 2.5 mm. For one or more embodiments, the anchor may have a thickness of 0.25 mm to 5 mm. For example, the anchor may have a thickness of 0.5 mm to 3 mm, or 0.75 mm to 2 mm.

For one or more embodiments, the plug, as disclosed herein, may be biodegradable. For example, the plug may degrade within a body in a period of time of 3 days to 180 days. For some applications the plug may degrade within the body in a period of time of 30 days to 70 days.

For one or more embodiments, the plug is formed from materials that may promote clotting. For some preferred embodiments, the plug includes collagen, gelatin, PEG, starch, or combinations thereof.

The cinch button, as disclosed here, may be resorbable, e.g., the cinch button may degrade within a body in a period of time of 30 days to 120 days. For some applications the cinch button may degrade within the body in a period of time of 60 days to 90 days. For one or more embodiments, the cinch button may include an ester, as discussed herein. For one or more embodiments, the cinch button may include a sugar, as discussed herein.

As discussed herein, the plug may have an undeformed state and a deformed state. The undeformed state is prior to the plug engaging an abluminal surface of the body lumen and/or a portion of the anchor as illustrated in FIG. 1. The deformed state is when the plug engages the abluminal surface of the body lumen and/or a portion of the anchor as illustrated in FIG. 2.

For various applications, the plug in the undeformed state may have differing shapes. For one or more embodiments, plug in the undeformed state may include one or more polyhedron, sphere, cylinder, tubular, cone, and combinations thereof For some preferred embodiments, the plug in the undeformed state may have a cylindrical shape.

For various applications the plug in the undeformed state may have differing dimensions. For one or more embodiments, the plug in the undeformed state may have a length within a range of from 0.5 cm to 5 cm; e.g, the plug in the undeformed state may have a length of 1 cm to 4 cm, or 1.5 cm to 3 cm. For one or more embodiments, plug in the undeformed state may have a width within a range of from 0.7 mm to 8 mm. For example, the plug in the undeformed state may have a width of 1 mm to 5 mm, or 1.5 mm to 3 mm. The plug may have a generally circular cross section or rectangular cross section, with a thickness in ranges similar to the width ranges.

Embodiments of the present disclosure provide a system for closing an opening in a body lumen. FIG. 9 illustrates a system 926 for closing an opening in a body lumen. The system 926 includes an insertion sheath 928, a device sheath 930 disposed in the insertion sheath 928, and the closure device 900. The plug 902, a portion of the suture 906, and the cinch button 916 can be releasably housed in the device sheath 930, where the anchor is releasably housed in the insertion sheath 928.

The system further includes a push member 932 disposed in the device sheath 930, where the push member 932 extends to advance the closure device 900 from the insertion sheath 928 and to apply the compressive axial force to the cinch button 616 in deploying the closure device 900. Push member 932 may be located, e.g., adjacent the cinch button 916 of the closure device 900.

Closure device 900 may include the plug 902, the anchor 904, the cinch button 916, and the suture 906 coupled to the anchor 904, where the suture 906 includes a plurality of elongate filaments 912 having a predefined cutting segment 914 for cutting the suture 906. The closure device 900 includes the cinch button 916 having the aperture 918, wherein the suture 906 is positioned through the aperture 918 such that the cinch button 916 can move longitudinally along the suture 906. The plurality of elongate filaments 912 of the predefined cutting segment 914 are bonded together to prevent their relative motion, as discussed herein. The anchor 904 may include a furrow and/or channel along a portion of the anchor. The furrow and/or channel may be located along a central portion of the anchor. The anchor 904 including the furrow and/or channel may be foldable within the insertion sheath 928.

For one or more embodiments, the system 926 may include a remote cutting mechanism 934 and can include a shearing-type cutting element to cut the suture along the predefined cutting segment. The remote cutting mechanism 934 is configured to cut the suture 906 along the predefined cutting segment 914 once the push member 932 compresses the plug 902 to the deformed state. The remote cutting mechanism 934 is illustrated in FIG. 9 as being positioned at a distal end of the push member 932. However, the remote cutting mechanism 934 may be separate from the push member 932.

For one embodiment, the remote cutting mechanism 934 further includes a shearing-type cutting element that can be actuated. The remote cutting mechanism 934 can be actuated manually when desired, or automatically upon completion of deformation of plug 902.

For one or more embodiments, the system 926 may include a handle 936. Handle 936 may include one or more control members, such as but not limited to, a slider 938. The one or more control members may be coupled the anchor 904 and may help control positioning of the anchor 904. Handle 936 may also include a number of different and/or alternative structural features.

For one or more embodiments, the system 926 may include one or more actuation members 940. The one or more actuation members 940 may be coupled to the insertion sheath 928, the suture 906, and/or the remote cutting mechanism 934. The one or more actuation members 940 may function to retract the insertion sheath 928, apply a tension force to the suture 906, apply the compressive axial force to the cinch button 916 in deploying the closure device 900, and/or activate the remote cutting mechanism 934 to cut the suture 906.

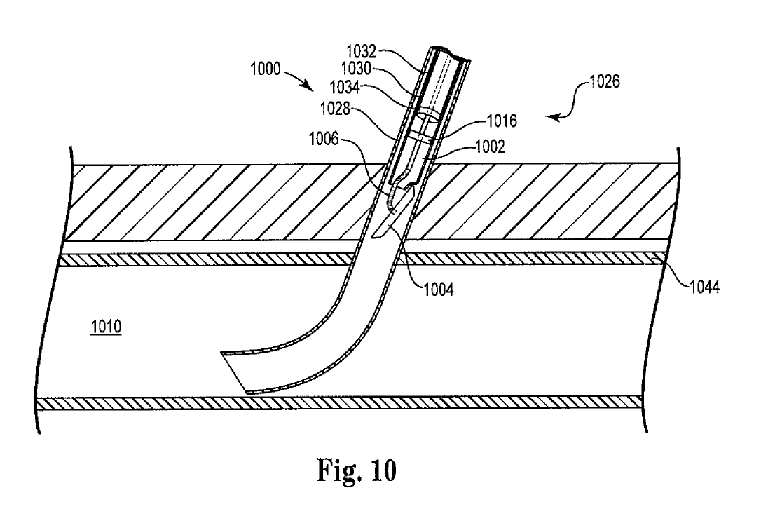

FIG. 10 illustrates the system 1026 disposed within the insertion sheath 1028. As illustrated in FIG. 10, the insertion sheath 1028 extends through the skin and the body vessel wall 1044 and into the body lumen 1010, e.g., the femoral artery. Deployment of the system 1026 may include the use of an obturator and/or dilator.

For some applications, the system 1026 including the closure device 1000 may be advanced through insertion sheath 1028 to a position where the anchor 1004 is advanced into the body lumen 1010. After and/or while being advanced out from the insertion sheath 1028, the anchor 1004 may be configured to shift and/or tilt to prepare for engagement with body lumen wall. The shifting and/or tilting may be accomplished in a number of different ways. In one embodiment, fluid expansion of the plug 1002 provides the energy to flip anchor 1004 around towards the desired position. In another embodiment, suture 1006 may be configured or otherwise be arranged in conjunction with the anchor 1004 so that the suture 1006 may be manipulated to cause the anchor 1004 to shift and/or tilt. For some applications, the suture 1006 may be wrapped and/or wound around one or more portions of the anchor.

For some applications, when the anchor 1004 is prepared for engagement with the body lumen wall 1044, device sheath 1030 and/or insertion sheath 1028 may be withdrawn, e.g., moved proximally, so that the anchor 1004 is positioned in a desired location, such as engaging or imminently prepared to engage the body lumen wall 1044. For one or more embodiments, the distal end of the insertion sheath 1028 can have a bevel, as seen in FIG. 10. The bevel can help orient the anchor 1002 axially and cause the anchor 1002 to be parallel with the body vessel wall 1044 just prior to engaging the body lumen wall 1044.

For some applications, the insertion sheath 1028 and/or the device sheath 1030 can be retracted, e.g., moved proximally, to provide a gap for deployment of the plug 1002. In other embodiments, insertion sheath 1028 is configured to deform the plug 1002 during deployment, allowing the plug 1002 to move outward for deployment whilst displacing a portion of insertion sheath 1028 out of the way, e.g., a shoehorn sheath.

After and/or while the anchor 1004 is positioned in the desired location, push member 1032 may be advanced, e.g., moved distally, so as to engage and apply a push force to the cinch button 1016 that can pull together and/or secure the anchor 1004 with the plug 1002. For one or more embodiments, the push force applied to the cinch button 1016 via the push member 1032 is sufficient to compress the plug 1002, e.g. transition the plug 1002 from the undeformed state to the deformed state. For one or more embodiments, the deformed plug 1002 engages the abluminal surface of the body vessel and/or a portion of the anchor 1004.

Once desirably situated, e.g., the anchor 1004 is engaging the adluminal surface of the body vessel wall 1044 and the plug 1002 is engaging the abluminal surface of the body vessel wall 1044 and/or a portion of the anchor 1004, the insertion sheath 1028 and/or the device sheath 1030 may be retracted to leave the closure device 1000 closing the opening in the body lumen 1010.

The excess suture, e.g., a portion of suture extending from the desirably situated closure device, may be removed by cutting the suture 1006 with the remote cutting mechanism 1034. As discussed herein, the remote cutting mechanism 1034 can be provided on the push member 1032. Alternatively, the remote cutting mechanism 1034 can be a separate device which is advanced after the closure device 1000 is desirably situated, to cut the excess suture.

In one or more embodiments, the present disclosure also includes a method of making a closure device for closing an opening in a body lumen. A method of making the closure device may include providing a suture having a plurality of elongate filaments and forming a predefined cutting segment along the suture by bonding the plurality of elongate filaments to prevent their relative motion. The method can include embedding a portion of the suture into an anchor to couple the suture to the anchor and positioning a plug over a length of the suture. The method can further include positioning a cinch button along the suture to couple the anchor to the plug.

For one or more embodiments, forming the predefined cutting segment can include applying a bonding agent that is biodegradable to the plurality of elongate filaments of the predefined cutting segment. For one or more embodiments, the bonding agent can be selected from the bonding agents as discussed herein.

For one or more embodiments, forming the predefined cutting segment can include applying compression in combination with at least one of the bonding agent, a solvent, and heat to the plurality of elongate filaments of the predefined cutting segment. Compression can be applied to the plurality of elongate filaments of the predefined cutting segment for period of time sufficient to minimize the available space between the plurality of elongate filaments. The application of the bonding agent, the solvent, and/or heat can bond the plurality of elongate filaments of the predefined cutting segment together to prevent their relative motion.

Examples of applying compression include, but are not limited to, a mold compression tool, an iris compression tool, a heat-shrink compression tool, an inflatable compression tool, and combinations thereof. Examples of the bonding agent and the solvent include the bonding agents and solvents discussed herein.

For one or more embodiments, forming the predefined cutting segment can include applying heat to the plurality of elongate filaments of the predefined cutting segment. Applying heat to the plurality of elongate filaments of the predefined cutting segment can include, but is not limited to, hot-air heating, ultrasonic heating, melding, lasers, infrared heating, electron-beam energy, contact heating, heated-bath heating, and combinations thereof. As discussed herein, heat can be applied alone or used in combination with compression, the bonding agent, and/or the solvent.

For one or more embodiments, forming the predefined cutting segment can include applying a solvent to the plurality of elongate filaments of the predefined cutting segment. Applying the solvent can include dissolving a portion of the plurality of elongate filaments of the predefined cutting segment. Applying the solvent can include softening of the surface of the filaments and/or making the filaments tacky so that they stick together. Applying the solvent can include applying a chemical modifier or cross-linking agent to aid in bonding. Once the portion of the plurality of elongate filaments are dissolved, they can be fused together by applying compression, as discussed herein, which bonds the plurality of elongate filaments together preventing their relative motion. Additionally, substantially all of the plurality of elongate filaments can be bonded together providing a single solid structure. The predefined cutting segment is allowed to dry for a time period sufficient to allow the solvent to evaporate. For one or more embodiments, the solvent can be selected from the solvents as discussed herein. The solvent can be applied alone or in combination with heat, the bonding agent, and/or compression.

For one or more embodiments, forming the predefined cutting segment includes applying tension in combination with at least one of the bonding agent, the solvent, and heat to the plurality of elongate filaments of the predefined cutting segment. Applying tension to the plurality of elongate filaments of the predefined cutting segment can include applying grasping mechanisms to each end of the plurality of elongate filaments, and pulling the grasping mechanisms apart to tension, i.e., neck, the plurality of elongate filaments. Direct or indirect grasping can be utilized. Alternatively, applying tension can include applying one or more grasping or compression mechanisms at one or more locations along the length of the plurality of elongate filaments, and using the grasping or compression mechanism(s) to tension the plurality of elongate filaments.

The present invention is particularly advantageous for facilitating cutting of multifilament suture, but can also be used to facilitate cutting of monofilaments that are so small and/or so flexible that they are difficult to reliably cut. For one or more embodiments, the cutting segment is rendered larger and/or stiffer, i.e., more rigid, by the present invention, to facilitate cutting of the suture.

In a further example, a porous monofilament material such as expanded polytetrafluoroethylene (PTFE) monofilament suture can be modified by the present invention for more reliable cutting within the scope of the invention. While PTFE is an example of a non-biodegradable suture material, a biodegradable suture which can have gaps, pockets, bubbles, pores, hard- and soft-sections, narrowings, or other structures that render it too flexible for reliably cutting the using a remote cutting mechanism, yet it may not have a typical multifilament arrangement, can be more reliably cut using the present invention.

For one or more embodiments, degradable closure devices are typically preferred. Alternatively, non-degradable devices can be used, utilizing various polymeric and/or metallic components as are known in the art, with the improvement of the present invention providing more reliable remote suture cutting.

It is to be understood that the above description has been made in an illustrative fashion, and not a restrictive one.

What is claimed:

1. A closure device for closing an opening in a body lumen comprising:
a plug;
an anchor;
a cinch button; and
a suture coupled to the anchor, the suture having a plurality of elongate filaments with a predefined cutting segment for cutting the suture, wherein the plurality of elongate filaments of the predefined cutting segment are bonded together to prevent their relative motion.

2. The closure device of claim 1, wherein a rigidity of the predefined cutting segment is greater than a rigidity of the suture that is not the predefined cutting segment.

3. The closure device of claim 1, wherein the cinch button includes an aperture, wherein the suture is positioned through the aperture such that the cinch button can move longitudinally along the suture to deform the plug.

4. The closure device of claim 1, wherein the predefined cutting segment includes a bonding agent that is biodegradable.

5. The closure device of claim 4, wherein the bonding agent is selected from the group consisting of polymers, sugars, adhesives, biological materials, and combinations thereof.

6. The closure device of claim 5, wherein the bonding agent is selected from the group consisting of polygycolic acid, polylactic acid, poly(lactic-co-glycolic acid), glycols, proteins, polyesters, monosaccharides, disaccharides, polysaccharides, polyanhydrides, cyanoacrylates, and combinations thereof combinations thereof.

7. The closure device of claim 1, wherein a distal end of the predefined cutting segment is positioned at a predetermined distance from a surface of the anchor.

8. The closure device of claim 1, wherein the predefined cutting segment has a first cross-sectional area that is the same as a second cross-sectional area of the suture that is not the predefined cutting portion, wherein the first cross-sectional area and the second cross-sectional area are perpendicular to a longitudinal axis of the suture.

9. The closure device of claim 1, wherein the predefined cutting segment has a first cross-sectional area that is less than a second cross-sectional area of the suture that is not the predefined cutting portion, wherein the first cross-sectional area and the second cross-sectional area are perpendicular to a longitudinal axis of the suture.

10. A system for closing an opening in a body lumen comprising:
an insertion sheath;
a device sheath releasably housed in the insertion sheath;
a closure device, the closure device including
a plug releasably housed in the device sheath;
an anchor for positioning in the body lumen releasably housed in the insertion sheath;
a cinch button positioned along the suture and is releasably housed in the device sheath; and
a suture coupled to the anchor, the suture having a plurality of elongate filaments with a predefined cutting segment for cutting the suture, where the plurality of elongate filaments of the predefined cutting segment are bonded together to prevent their relative motion; and
a push member disposed in the device sheath, wherein the push member is configured to deform the plug.

11. The system of claim 10, wherein the cinch button includes an aperture, wherein the suture is positioned through the aperture such that the cinch button can move longitudinally along the suture.

12. The system of claim 10, further including a remote cutting mechanism, wherein the remote cutting mechanism is configured to cut the suture along the predefined cutting segment.

13. The system of claim 12, wherein the remote cutting mechanism includes a shearing-type cutting element to cut the suture along the predefined cutting segment.

14. A method of making a closure device for closing an opening in a body lumen, comprising:
providing a suture having a plurality of elongate filaments;
forming a predefined cutting segment along the suture by bonding the plurality of elongate filaments together to prevent their relative motion;
embedding the suture into an anchor to couple the suture to the anchor;
positioning a plug over a portion of the suture; and
positioning a cinch button along the suture to couple the plug to the anchor.

15. The method of claim 14, wherein forming the predefined cutting segment includes applying a bonding agent to the plurality of elongate filaments of the predefined cutting segment.

16. The method of claim 15, wherein applying the bonding agent includes selecting the bonding agent from the group consisting of biodegradable polymers, sugars, biological materials, proteins, adhesives, and combinations thereon.

17. The method of claim 14, wherein forming the predefined cutting segment includes applying compression in combination with at least one of: a bonding agent, a solvent, and heat to the plurality of elongate filaments of the predefined cutting segment.

18. The method of claim 14, wherein forming the predefined cutting segment includes applying heat to the plurality of elongate filaments of the predefined cutting segment.

19. The method of claim 14, wherein fanning the predefined cutting segment includes applying a solvent to the plurality of elongate filament of the predefined cutting segment.

20. The method of claim 14, wherein forming the predefined cutting segment includes applying tension in combination with at least one of: a bonding agent, a solvent, and heat to the plurality of elongate filaments of the predefined cutting segment.
